(19) United States
(12) Patent Application Publication
(10) Pub. No.: US 2026/0234848 A1
(43) Pub. Date: Aug. 13, 2026

(54) WATER-DISSOLVABLE FIBROUS ARTICLE HAVING APERTURES

(71) Applicant: THE PROCTER & GAMBLE COMPANY, Cincinnati, OH (US)

(72) Inventors: Sarah Noelle ABSHER, Cincinnati, OH (US); Ronald Joseph ZINK, II, Cincinnati, OH (US); Michael Sean PRATT, Cincinnati, OH (US); Shawn Christopher PALLOTTA, Maineville, OH (US); Jennifer Elaine HILVERT, Cincinnati, OH (US); Mark William HAMERSKY, Hamilton, OH (US)

(21) Appl. No.: 19/530,766

(22) Filed: Feb. 5, 2026

Related U.S. Application Data
(60) Provisional application No. 63/755,357, filed on Feb. 7, 2025.

(51) Int. Cl. D04H 1/4282; D04H 1/413; D06C 23/04

(52) U.S. Cl. CPC D04H 1/4282; D04H 1/413; D06C 23/04; D10B 2321/00; D10B 2401/024

(57) ABSTRACT

A water-dissolvable fibrous article. The article is formed by a mixture comprising polymeric structurant, one or more high melting point fatty material having a carbon chain length C12-C22 or mixtures thereof, and a cationic surfactant. The water-dissolvable fibrous article has one or more apertures. The apertures have one or more of the following properties: an Aperture Elliptical Length of from about 0.1 mm to about 10 mm, as measured according to the Aperture Characterization Test Method and/or an Aperture Elliptical Area of from about 0.5 mm² to about 15 mm², as measured according to the Aperture Characterization Test Method.

### FIELD OF THE INVENTION

The present disclosure relates to a water-dissolvable fibrous article, wherein the article is formed by a mixture comprising, by weight of the article: from about 6% to about 30% of a polymeric structurant such as polyvinylpyrrolidone and its copolymers; from about 10 wt % to about 85 wt % of one or more high melting point fatty material having a carbon chain length C12-C22 or mixtures thereof, wherein the melting point is above 25° C.; and from about 1 wt % to about 60 wt % of a cationic surfactant; wherein the water-dissolvable fibrous article has one or more apertures which exhibit at least one of the following properties: a) an Aperture Elliptical Length of from about 0.1 mm to about 10 mm as measured according to the Aperture Characterization Test Method described herein; and b) an Aperture Elliptical Area of from about 0.5 mm² to about 15 mm² as measured according to the Aperture Characterization Test Method described herein. The present disclosure provides a water-dissolvable fibrous article with apertures, even when the article may exhibit sticky behavior, for example, articles sticking to surfaces of process equipment during their making. The article of the present disclosure provides improved dissolution and/or improved water penetration into the article, especially for articles with increased size or weight, for example, improve dissolution of less than 10 hand strokes for the article with increased basis weight of 800 gsm or more, preferably 1400 gsm or more. Additionally, the water-dissolvable fibrous articles of the present inventing may exhibit reduced tendency to stick to surfaces of process equipment during the aperture formation.

### BACKGROUND OF THE INVENTION

Many personal care and other consumer products in the market today are sold in liquid form. While widely used, liquid products often have tradeoffs in terms of packaging, storage, transportation, and convenience of use. Liquid consumer products typically are sold in bottles which add cost as well as packaging waste, much of which ends up in land-fills.

Hair Care products in the form of a dissolvable solid articles/structures present an attractive form to consumers. Market executions of dissolvable solid structures may include, dissolvable films, compressed powders in a solid, fibrous article/structures, porous foams, soluble deformable solids, powders, etc.

For example, PCT Application Publication No. WO2023034763A1 discloses dissolvable solid fibrous article/structure comprising polyvinylpyrrolidone, a cationic surfactant, and one or more high melting point fatty material having a carbon chain length C12-C22 or mixtures thereof.

Another example can be PCT Application Publication No. WO2016057353A1 which discloses dissolvable fibrous article/structures with apertures. In this 2016 publication, it is said that such soluble fibrous structures with apertures, are at a minimum perceived by consumers as exhibiting improved dissolution properties and/or actually exhibit improved dissolution properties. This 2016 publication discloses a variety of polymers such as hydroxyethyl cellulose and polyvinyl alcohol as a filament forming material.

There still exists a need for forming apertures on dissolvable fibrous article containing a polymeric structurant such as polyvinylpyrrolidone, a cationic surfactant, and a high melting point fatty compound, such that the articles are at a minimum perceived by consumers as exhibiting improved dissolution properties and/or actually exhibit improved dissolution properties.

However, it has been surprisingly found by the present inventors that such dissolvable fibrous article containing a polymeric structurant such as polyvinylpyrrolidone, a cationic surfactant, and a high melting point fatty compound, is very sticky, thus, it is difficult to form apertures on such articles.

### SUMMARY OF THE INVENTION

The present disclosure is directed to a water-dissolvable fibrous article, wherein the article is formed by a mixture comprising, by weight of the article:
from about 6% to about 30%, preferably from about 8% to about 23%, more preferably form about 10% to about 20% of a polymeric structurant;
from about 10 wt % to about 85 wt % of one or more high melting point fatty material having a carbon chain length C12-C22 or mixtures thereof, wherein the melting point is above 25° C.; and
from about 1 wt % to about 60 wt % of a cationic surfactant;
wherein the water-dissolvable fibrous article has one or more apertures which exhibit at least one of the following properties, preferably both of the following properties:
   a) an Aperture Elliptical Length of from about 0.1 mm to about 10 mm, preferably from about 0.2 mm to about 6 mm, as measured according to the Aperture Characterization Test Method described herein; and
   b) an Aperture Elliptical Area of from about 0.5 mm² to about 15 mm², preferably from about 0.5 mm² to about 7 mm² as measured according to the Aperture Characterization Test Method described herein.
      Preferably, the apertures exhibit the following additional property:
c) an Aperture Elliptical Percentage of from about 0.1% to about 50%, more preferably from about 0.5% to about 20% as measured according to the Aperture Characterization Test Method described herein.

Preferably, the article exhibits a Stickiness Index of from about 20% to about 100%, preferably from about 30% to about 100%, more preferably from about 40% to about 100%, still more preferably from about 50% to about 100%, even more preferably from about 60% to about 99% as measured according to the Stickiness Index Test Method described herein.

Preferably, the article is comprised of more than 1 ply with the apertures formed through article surfaces.

Preferably, the apertures are formed in a pattern registered to the shape of the article.

Preferably, the apertures are formed by a non-sticky material selected from the group consisting of: silicone polymer, polyurethane, and blend of them, and wherein the material has a Shore A hardness of 95 or below.

Preferably, the article has a basis weight of 800 gsm or more, preferably 1400 gsm or more.

Preferably, the article comprises from about 40 wt % to about 65 wt % of one or more high melting point fatty alcohol material having a carbon chain length C12-C22 or mixtures thereof, wherein the melting point is above 25° C.; and from about 15 wt % to about 30 wt % of a cationic surfactant.

Preferably, the article dissolves in less than 15 strokes, preferably less than 10 strokes of the Hand Dissolution Method.

Preferably, the article contains a particulate material, wherein the particulate material is integrated into the fibrous structure, and/or is between plies of the article.

Preferably, the polymeric structurant is one selected from polyvinylpyrrolidone, its copolymers, and combinations thereof.

Preferably, the polymeric structurant is selected from the group consisting of: polyacrylic acid and its copolymers, polyacrylamide and its copolymers, polyvinylmethyl ether, polyethyleneimine, polymethacrylic acid, polyN-isopropyl acrylamide, polyN-N-dimethylacrylamide, poly(2-ethyl-2-oxazoline), polyoxazoline, polyvinyl oxazoline, polyalkyl oxazoline, polyaryl oxazoline, polyvinyloxazolidone, polyvinyl caprolactam, polystyrene sulfonate, polyvinyl formamide, polyvinyl amine, alkylated polyvinyl pyrrolidone, poly vinyl caprolactam, polyvinyl valerolactam, polyvinyl imidazole, polyacrylic acid, polyacrylamide, polymethacrylamide, polydimethacrylamide, polyalkylaminomethacrylate, and polyalkylaminomethacrylamide, and combinations thereof, more preferably the polymeric structurant is selected from the group consisting of: polydimethylacrylamide, polyalkyl oxazoline, poly (2-ethyl-2 oxazoline), polyaryl oxazoline, and combinations thereof.

It has been surprisingly found by the present inventors that such dissolvable fibrous article containing a polymeric structurant such as polyvinylpyrrolidone, a cationic surfactant, and a high melting point fatty compound, is very sticky immediately after processing. Thus, it has been surprisingly found by the present inventors that it is difficult to form apertures on such articles containing a polymeric structurant such as polyvinylpyrrolidone, a cationic surfactant, and a high melting point fatty compound.

The present disclosure provides a water-dissolvable fibrous article with apertures, even when the article may exhibit sticky behavior, for example, articles sticking to surfaces of process equipment during their making. With such apertures, the article of the present disclosure provides improved dissolution and/or improved water penetration into the article, especially for articles with increased size or weight, for example, improve dissolution of less than 10 hand strokes for the article with increased basis weight of 800 gsm or more, preferably 1400 gsm or more. Additionally, the water-dissolvable fibrous articles of the present inventing may exhibit reduced tendency to stick to surfaces of process equipment during the aperture formation.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a schematic representation of an example of a process for making fibrous elements; and

FIG. 2 is a schematic representation of an example of a die with a magnified view used in the process of FIG. 1.

FIG. 3 is an example of a fibrous article containing filaments.

FIG. 4 is an example of a fibrous articles comprising apertures according to the present disclosure.

FIG. 5 is another example of a fibrous articles comprising apertures according to the present disclosure.

FIG. 6 is another example of a fibrous articles comprising apertures according to the present disclosure.

FIG. 7 is a schematic representation of an aperturing process according to the present disclosure.

FIG. 8 is a perspective view of an example of a portion of a rotary knife aperturing apparatus.

FIG. 9 is a perspective view of an example of a pinning aperturing apparatus.

FIG. 10A is a perspective view of an example of an aperturing pattern to be used in a rotary die cutting device.

FIG. 10B is a perspective view of an example of a roller designed to receive aperture pattern inserts.

FIG. 10C is an example of a die cutting device containing a compliant aperturing insert.

FIG. 10D is a top view of an example of an aperturing insert with a pattern of protrusions registered to the shape of the article.

### DETAILED DESCRIPTION OF THE INVENTION

### Definitions

As used herein, The Water-Dissolvable Fibrous Article may be referred to herein as “the (Water-)Dissolvable Fibrous Article”, “the (Water-)Dissolvable Solid Article”, “the (Water-)Dissolvable Article”, “Fibrous Article” or “the Article”.

As used herein, “dissolvable” means that the Dissolvable Article is completely soluble in water or it provides a uniform dispersion upon mixing in water according to the hand dissolution test. The Dissolvable Article has a hand dissolution value of from about 1 to about 30 strokes, alternatively from about 2 to about 25 strokes, alternatively from about 3 to about 20 strokes, and alternatively from about 4 to about 15 strokes, as measured by the Hand Dissolution Method.

As used herein, “flexible” means a Dissolvable Article meets the distance to maximum force values discussed herein.

“Fibrous article/structure” as used herein means an article/structure that comprises one or more fibrous elements and optionally, one or more particles. The fibrous article/structure as described herein can mean an association of fibrous elements and optionally, particles that together form an article/structure, such as a unitary article/structure, capable of performing a function.

The fibrous article/structure of the present disclosure may have a multi-ply fibrous article/structure which comprises two or more different fibrous article/structure plies. Each ply may be the same as or different from other ply.

A layer comprising fibrous elements may sometimes be referred to as a ply. A ply may be a fibrous article/structure which may be homogeneous or layered as described herein.

The single-ply fibrous article/structure or a multi-ply fibrous article/structure comprising one or more fibrous article/structure plies as described herein may exhibit a basis weight of less than 5000 g/m² as measured according to the Basis Weight Test Method described herein. For example, the single- or multi-ply fibrous article/structure according to the present disclosure may exhibit a basis weight of greater than 10 g/m² to about 5000 g/m² and/or greater than 10 g/m² to about 3000 g/m² and/or greater than 10 g/m² to about 2000 g/m² and/or greater than 300 g/m² to about 3000 g/m² and/or greater than 500 g/m² to about 2000 g/m² as measured according to the Basis Weight Test Method.

In one example, the fibrous article/structure of the present disclosure is a “unitary fibrous article/structure.”

“Unitary fibrous article/structure” as used herein is an arrangement comprising a plurality of two or more and/or three or more fibrous elements that are inter-entangled or otherwise associated with one another to form a fibrous article/structure and/or fibrous article/structure plies. A unitary fibrous article/structure of the present disclosure may be one or more plies within a multi-ply fibrous article/structure. In one example, a unitary fibrous article/structure of the present disclosure may comprise three or more different fibrous elements. In another example, a unitary fibrous article/structure of the present disclosure may comprise two or more different fibrous elements.

“Article” as used herein refers to a consumer use unit, a consumer unit dose unit, a consumer use saleable unit, a single dose unit, or other use form comprising a unitary fibrous dissolvable solid article/structure and/or comprising one or more fibrous article/structures of the present disclosure.

“Fibrous element” as used herein means an elongated particulate having a length greatly exceeding its average diameter, i.e., a length to average diameter ratio of at least about 10. A fibrous element may be a filament or a fiber. In one example, the fibrous element is a single fibrous element rather than a yarn comprising a plurality of fibrous elements.

The fibrous elements of the present disclosure may be spun from a filament-forming composition also referred to as fibrous element-forming compositions via suitable spinning process operations, such as meltblowing, spunbonding, electro-spinning, and/or rotary spinning.

The fibrous elements of the present disclosure may be monocomponent (single, unitary solid piece rather than two different parts, like a core/sheath bicomponent) and/or multicomponent. For example, the fibrous elements may comprise bicomponent fibers and/or filaments. The bicomponent fibers and/or filaments may be in any form, such as side-by-side, core and sheath, islands-in-the-sea and the like.

“Filament” as used herein means an elongated particulate as described above that exhibits a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.). Filaments are typically considered continuous or substantially continuous in nature. Filaments are relatively longer than fibers. Non-limiting examples of filaments include meltblown and/or spunbond filaments.

“Fiber” as used herein means an elongated particulate as described above that exhibits a length of less than 5.08 cm (2 in.) and/or less than 3.81 cm (1.5 in.) and/or less than 2.54 cm (1 in.). Fibers are typically considered discontinuous in nature. Non-limiting examples of fibers include staple fibers produced by spinning a filament or filament tow and then cutting the filament or filament tow into segments of less than 5.08 cm (2 in.) thus producing fibers. Therefore, references to filaments herein also include fibers made from such filaments unless otherwise noted. Fibers are typically considered discontinuous in nature relative to filaments, which are considered continuous in nature.

“Filament-forming composition” and/or “fibrous element-forming composition” as used herein means a composition that is suitable for making a fibrous element of the present disclosure such as by meltblowing and/or spunbonding. The filament-forming composition comprises one or more filament-forming materials that exhibit properties that make them suitable for spinning into a fibrous element. In one example, the filament-forming material comprises a polymer. In addition to one or more filament-forming materials, the filament-forming composition may comprise one or more additives, for example one or more active agents. In addition, the filament-forming composition may comprise one or more polar solvents, such as water, into which one or more, for example all, of the filament-forming materials and/or one or more, for example all, of the active agents are dissolved and/or dispersed prior to spinning a fibrous element, such as a filament from the filament-forming composition.

As used herein, “porous” means that the Dissolvable Article has spaces, voids or interstices, (generally referred to herein as “pores”) provided by the microscopic complex three-dimensional configuration, that provide channels, paths or passages through which a liquid or water can flow.

As used herein, “porosity” and “percent porosity” are used interchangeably and each refers to a measure of void volume of the Dissolvable Article and is calculated as

$$[ 1 - ( [ basis weight of Dissolvable Article ] / [ thickness of Dissolvable Article \times density of the bulk , dried material ] ) ] \times 100 \%$$

with the units adjusted so they cancel and multiplied by 100% to provide percent porosity.

The term “molecular weight” or “Molecular weight” refers to the weight average molecular weight unless otherwise stated. Molecular weight is measured using industry standard method, gel permeation chromatography (“GPC”).

As used herein, the articles including “a” and “an” when used in a claim, are understood to mean one or more of what is claimed or described.

As used herein, the terms “include,” “includes,” and “including,” are meant to be non-limiting.

The methods disclosed in the Test Methods Section of the present application should be used to determine the respective values of the parameters of Applicants' inventions, including those discussed in the Dissolvable Articles-Physical Characteristics section below.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Water-Dissolvable Fibrous Articles

The water-dissolvable fibrous article of the present disclosure is formed by a mixture comprising, by weight of the article: from about 6% to about 30% of a polymeric structurant; from about 10 wt % to about 85 wt % of one or more high melting point fatty material having a carbon chain length C12-C22 or mixtures thereof, wherein the melting point is above 25° C.; and from about 1 wt % to about 60 wt % of a cationic surfactant; wherein the water-dissolvable fibrous article has one or more apertures which exhibit at least one of the following properties: a) an Aperture Elliptical Length of from about 0.1 mm to about 10 mm as measured according to the Aperture Characterization Test Method described herein; and b) an Aperture Elliptical Area of from about 0.5 mm² to about 15 mm² as measured according to the Aperture Characterization Test Method described herein.

The dissolvable article is preferably a fibrous article formed by a plurality of fibrous elements wherein the fibrous elements are formed by the above mixture, preferably wherein the mixture is homogeneous.

The dissolvable article is preferably solid and a personal care product, more preferably hair care product, still more preferably rinse-off hair care product, even more preferably rinse-off hair care product containing non-sulfate surfactant.

The dissolvable article may contain particulates. The dissolvable article may have an aesthetic feature selected from the group consisting of printing, embossing, texture, colored and mixtures thereof.

### Apertures

The dissolvable article comprises one or more apertures. Such apertures exhibit at least one of the following properties, preferably both of the following properties:
a) an Aperture Elliptical Length of from about 0.1 mm to about 10 mm, preferably from about 0.2 mm to about 6 mm, as measured according to the Aperture Characterization Test Method described herein; and
b) an Aperture Elliptical Area of from about 0.5 mm² to about 15 mm², preferably from about 0.5 mm² to about 7 mm² as measured according to the Aperture Characterization Test Method described herein.

The above Aperture Elliptical Length is larger than the Fiber Elliptical Length created in the fibrous article, whereas the Fiber Elliptical Length is from about 0.07 mm to 0.05 mm. The above Aperture Elliptical Area is larger than the Fiber Elliptical Area created in the fibrous article, where the Fiber Elliptical Area is from about 0.004 mm² to 0.04 mm².

Apertures preferably exhibit the following additional property:
c) an Aperture Elliptical Percentage of from about 0.1% to about 50%, more preferably from about 0.5% to about 20% as measured according to the Aperture Characterization Test Method described herein.

The present disclosure provides a water-dissolvable fibrous article with apertures, even when the article may exhibit sticky behavior, for example, articles sticking to surfaces of process equipment during their making. By having such apertures, the article provides improved dissolution and/or increased water penetration into the article, especially for articles with increased size or weight, for example, improve dissolution of less than 10 hand strokes for the article with increased basis weight of 800 gsm or more, preferably 1400 gsm or more. Without being bound by theory, the inventors believe improved dissolution and/or the increased water penetration into the article may be achieved by the increased exposed surface area and access to the center layers of in the article by the dissolving water. Additionally, the water-dissolvable fibrous articles of the present inventing may exhibit reduced tendency to stick to surfaces of process equipment during the aperture formation.

With such apertures, the fibrous articles, for example soluble fibrous articles, are at a minimum perceived by consumers as exhibiting improved dissolution properties and/or actually exhibit improved dissolution properties than fibrous articles without apertures. In addition to the improved dissolution properties, the apertures within the fibrous articles of the present disclosure may provide bonding functions for bonding two or more plies of the fibrous article together. Furthermore, the present disclosure offers an opportunity to impart appealing visual and tactile aesthetics, improved softness, lower modulus, more flexible consumer feel, and higher levels of elongation to the fibrous articles. Additionally, the apertures provided within the fibrous article have been found to provide a means to change the mechanical properties of the fibrous article. In particular, the modulus of the fibrous article may be reduced; leading to a more flexible fibrous article suitable for cooperation with product dispensing apparatuses and further may be experienced by the end user as having improved product handling and softness.

“Aperture” as used herein means an opening or void or indentation in a fibrous article which is distinct from the surrounding fibrous article. In one example, an aperture may comprise any feature where there is a localized disruption of the fibrous article. In one example, an aperture may comprise a local indentation or localized disruption of the basis weight, thickness, or caliper of the fibrous article. In another example, an aperture may be an opening in a fibrous article wherein the opening passes substantially or completely through both generally planar surfaces of the fibrous article, through one generally planar surface of the fibrous article, or even through neither planar surface of the fibrous article. In another example, an aperture may be an opening in the fibrous article wherein there is a complete opening, partial opening, or even no apparent opening. In still another example, an aperture may comprise a feature which is an embossment in the fibrous article. In even another example, an aperture is an internal feature to a fibrous article and/or multi-ply fibrous article wherein for example the aperture feature may be present on an internal ply of a multi-ply fibrous article. In even yet another example, an aperture comprises an opening or void or indentation in a fibrous article wherein the opening or void or indentation is a non-random and/or designed and/or fabricated opening, void, or indentation rather than a random pore that exists between and/or amongst fibrous elements of a fibrous article resulting from the collection and inter-entangling of fibrous elements on a collection device.

Non-limiting examples of apertures within fibrous articles of the present disclosure are shown in FIG. 4 through FIG. 6.

In another example of the present disclosure, the fibrous article comprises one or more apertures and thus is an apertured fibrous article. In one example, the fibrous article comprises a plurality of apertures. The apertures may be arranged in a pattern, for example a repeating pattern, such as a non-random, repeating pattern, and/or a non-repeating pattern.

Apertures within the apertured fibrous article of the present disclosure may be of virtually any shape and size. In one example, the apertures within the apertured fibrous articles are generally round or oblong shaped, in a regular pattern of spaced apart openings. In one example, the fibrous article comprises two or more apertures that are spaced apart from one another at a distance of from about 0.2 mm to about 100 mm and/or from about 0.5 mm to about 10 mm.

Aperturing of fibrous articles, for example soluble fibrous articles, can be accomplished by any number of techniques. For example, aperturing can be accomplished by various processes involving bonding and stretching, such as those described in U.S. Pat. Nos. 3,949,127 and 5,873,868. In one embodiment, the apertures may be formed by forming a plurality of spaced, melt stabilized regions, and then ring-rolling the web to stretch the web and form apertures in the melt stabilized regions, as described in U.S. Pat. Nos. 5,628,097 and 5,916,661, both of which are hereby incorporated by reference herein. In another embodiment, apertures can be formed in a multilayer, fibrous article configuration by the method described in U.S. Pat. Nos. 6,830,800 and 6,863,960 which are hereby incorporated herein by reference. Still another process for aperturing webs is described in U.S. Pat. No. 8,241,543 entitled “Method And Apparatus For Making An Apertured Web”, which is hereby incorporated herein by reference. Non-limiting examples of processes for imparting apertures to a fibrous article of the present disclosure include embossing, rodding, rotary knife aperturing, pinning, die cutting, die punching, needle punching, knurling, crush cutting, shear cutting, pneumatic forming, hydraulic forming, laser cutting, and tufting. In one example, the fibrous article of the present disclosure comprises pinning-imparted apertures. In another example, the fibrous article of the present disclosure comprises rodding-imparted apertures. In another example, the fibrous article of the present disclosure comprises rotary knife aperturing-imparted apertures. In still another example, the fibrous article of the present disclosure may comprise apertures that have been imparted to the fibrous article by different types of aperturing processes.

In one example, apertures may be imparted to a fibrous article during forming of the fibrous article on a collection device, such as a patterned belt, that has features, for example depressions and/or protrusions that impart apertures to the fibrous article upon the fibrous elements contacting the collection device during formation.

In another example, the fibrous article may exhibit different regions, such as different regions of basis weight, density and/or caliper. In yet another example, the fibrous article may comprise texture on one or more of its surfaces. A surface of the fibrous article may comprise a pattern, such as a non-random, repeating pattern. The fibrous article may be embossed with an emboss pattern. In another example, the fibrous article may comprise apertures. The apertures may be arranged in a non-random, repeating pattern.

In one example, the fibrous article may comprise discrete regions of fibrous elements that differ from other parts of the fibrous article. Non-limiting examples of different regions within fibrous articles are described in U.S. Published Patent Application Nos. 2013/017421 and 2013/0167305 incorporated herein by reference.

### Stickiness Index

In one example, the articles of the present disclosure may exhibit a Stickiness Index of greater than about 20% and/or greater than about 30% and/or greater than about 40% and/or greater than about 50% and/or greater than about 60% as measured according to the Stickiness Index Test Method described herein. In one example, the articles of the present disclosure may exhibit a Stickiness Index of from about 20% to about 100% and/or from about 30% to about 100% and/or from about 40% to about 100% and/or from about 50% to about 100% and/or from about 60% to about 99% as measured according to the Stickiness Index Test Method described herein.

Dissolvable articles having lower Stickiness Index has reduced adhesion and tackiness, thus, it's possible to form apertures on such articles and/or apertures can be made with any conventional method/equipment on such articles without the articles sticking to process equipment. Dissolvable articles having higher Stickiness Index are more sticky, thus, it's difficult to form apertures on such articles and need specific aperture characterization of the present disclosure.

### Polymeric Structurant, Especially PVP Polymeric Structurant

The article comprises a polymeric structurant at a level of from about 6% to about 30%, preferably from about 8% to about 23%, more preferably form about 10% to about 20% by weight of the article.

Preferably, the polymeric structurant is one selected from polyvinylpyrrolidone (PVP), its copolymers, and combinations thereof (hereinafter also called as “PVP polymeric structurant”). Such PVP polymeric structurant is included in the article, at a level of preferably from about 6% to about 30%, more preferably from about 8% to about 23%, still more preferably form about 10% to about 20% by weight of the article.

Preferably, the article comprises:
from about 1% to about 10% of a first PVP polymeric structurant having a molecular weight of from about 1,000,000 g/mol to about 4,000,000 g/mol, preferably from about 1,500,000 g/mol to about 3,500,000 g/mol; and
from about 10% to about 20% of a second PVP polymeric structurant having a molecular weight of from about 50,000 g/mol to about 500,000 g/mol, preferably from about 65,000 g/mol to about 395,000 g/mol.

In the present disclosure, it is preferred the weight ratio between the first and second PVP polymeric structurants is from about 1:1 to about 1:100, more preferably from about 1:1 to about 1:50, still more preferably from about 1:1 to about 1:10.

In the present disclosure, it is preferred that the weight ratio between the first and second PVP polymeric structurants is in a range such that the combined average molecular weight of the first and second polymeric structurants is from about 300,000 g/mol to about 3,000,000 g/mol, more preferably from about 350,000 g/mol to about 2,000,000 g/mol, more preferably from about 400,000 g/mol to about 1,000,000 g/mol and most preferably from about 500,000 g/mol to about 1,000,000 g/mol.

In the present disclosure, it is preferred that the amounts of the first and second PVP polymeric structurants, the molecular weights of the first and second PVP polymeric structurants, and the weight ratio between the first and second PVP polymeric structurants are selected such that the mixture in melt at 85° C. has a viscosity of from about 0.3 Pa's to about 10 Pa s, preferably from about 3 Pa's to about 8 Pa s.

The first and second PVP polymeric structurants are water-soluble polymers. As used in the present disclosure, the term “water-soluble polymer” is broad enough to include both water-soluble and water-dispersible polymers, and is defined as a polymer with a solubility in water, measured at 25° C., of at least about 0.1 gram/liter (g/L). In some embodiments, the polymers have a solubility in water, measured at 25° C., of from about 0.1 gram/liter (g/L). to about 500 grams/liter (g/L). (This indicates production of a macroscopically isotropic or transparent, colored or colorless solution). The polymers for making these solids may be of synthetic or natural origin and may be modified by means of chemical reactions. They may or may not be film-forming. These polymers should be physiologically acceptable, i.e., they should be compatible with the skin, mucous membranes, the hair and the scalp. The terms “water-soluble polymer” and “polymer structurant” are used interchangeably herein. Furthermore, whenever the singular term “polymer” is stated, it should be understood that the term is broad enough to include one polymer or a mixture of more than one polymer. For instance, if a mixture of polymers is used, the polymer solubility as referred to herein would refer to the solubility of the mixture of polymers, rather than to the solubility of each polymer individually.

The first and second PVP polymeric structurants are selected from the group consisting of: polyvinylpyrrolidone and its copolymers, and mixtures thereof. More preferably, the first and second PVP polymeric structurants are polyvinylpyrrolidone.

As used herein, “vinyl pyrrolidone copolymer” (and “copolymer” when used in reference thereto) refers to a polymer of the following structure (I):

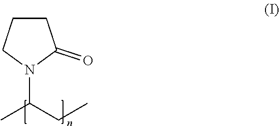

In structure (I), n is an integer such that the polymeric structurant has the degree of polymerization such that it possesses characteristics described herein. For purposes of clarity, the use of the term “copolymer” is intended to convey that the vinyl pyrrolidone monomer can be copolymerized with other non-limiting monomers such as vinyl acetate, alkylated vinyl pyrrolidone, vinyl caprolactam, vinyl valerolactam, vinyl oxazoline, 2-ethyl-2oxazoline, vinyl imidazole, acrylic acid, methacrylate, acrylamide, methacrylamide, dimethacrylamide, alkylaminomethacrylate, and alkylaminomethacrylamide monomers.

For example, suitable polymers for use are PVP K120 from Ashland having a weight average molecular weight of about 3,470,000 g/mol, PVP K90 from Ashland having a weight average molecular weight of 1,570,000 g/mol, PVP K60 from Jarchem having a weight average molecular weight of 396,000 g/mol, PVP K30 from Ashland having a weight average molecular weight of 66,800 g/mol. Additional suitable polymers include copolymers of polyvinylpyrrolidone, such as Ganex® or PVP/VA (weight average molecular weight of about 50,000 g/mol) copolymers from Ashland Inc., also performed as suitable structurants but a higher level was utilized to be effective due to their lower weight average molecular weight.

### Other Polymeric Structurant

The polymeric structurant useful herein also includes other polymers than the above PVP polymeric structurant. Such other polymeric structurant can be used with or without the above PVP polymeric structurant. When such other polymeric structurant are used with PVP polymeric structurant, such other polymeric structurant may be included such that the total weight of the above PVP polymeric structurant and such other polymeric structurant is up to about 30% by weight of the article.

Such other polymeric structurant useful herein are water-soluble polymers.

Non-limiting examples of such water-soluble polymers can include polyacrylic acid and its copolymers, polyacrylamide and its copolymers, polyvinylmethyl ether, polyethyleneimine, polymethacrylic acid, other water soluble acrylic polymers such as polyN-isopropyl acrylamide, polyN-N-dimethylacrylamide, poly(2-ethyl-2-oxazoline), polyoxazoline, polyvinyloxazolidone polyalkyloxazolidone, polyaryloxazoline, polyvinyl caprolactam, polystyrene sulfonate, polyvinyl formamide, polyvinyl amine, alkylated polyvinyl pyrrolidone, poly vinyl caprolactam, polyvinyl valerolactam, polyvinyl imidazole, polyacrylic acid, polyacrylamide, polymethacrylamide, polydimethacrylamide, polyalkylaminomethacrylate, and polyalkylaminomethacrylamide, and combinations thereof.

Preferably, such water-soluble polymers are, polydimethylacrylamide, polyalkyl oxazoline), poly(2-ethyl-2 oxazoline), polyaryloxazoline, and combinations thereof.

### Cationic Surfactant

The article further comprises a cationic surfactant at a level of from about 1 wt % to about 60 wt %, alternatively from about 10 wt % to about 50 wt %, alternatively from about 20 wt % to about 40 wt % by weight of the article.

Cationic surfactant useful herein can be one cationic surfactant or a mixture of two or more cationic surfactants. The cationic surfactant can be selected from the group consisting of, but not limited to: a mono-long alkyl quaternized ammonium salt; a combination of a mono-long alkyl quaternized ammonium salt and a di-long alkyl quaternized ammonium salt; a mono-long alkyl amine; a combination of a mono-long alkyl amine and a di-long alkyl quaternized ammonium salt; and a combination of a mono-long alkyl amine and a mono-long alkyl quaternized ammonium salt, a tertiary amine and combinations thereof.

### Mono-Long Alkyl Amine

Mono-long alkyl amine useful herein are those having one long alkyl chain of from 12 to 30 carbon atoms, alternatively from 16 to 24 carbon atoms, alternatively from 18 to 22 alkyl group. Mono-long alkyl amines useful herein also include mono-long alkyl amidoamines. Primary, secondary, and tertiary fatty amines are useful.

Suitable for use in the dissolvable article are tertiary amido amines having an alkyl group of from about 12 to about 22 carbons. Exemplary tertiary amido amines include: stearamidopropyldimethylamine, stearamidopropyldiethylamine, stearamidoethyldiethylamine, palmitamidopropyldimethylamine, stearamidoethyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present disclosure are disclosed in U.S. Pat. No. 4,275,055, Nachtigal, et al.

These amines can be used in combination with acids such as l-glutamic acid, lactic acid, hydrochloric acid, malic acid, succinic acid, acetic acid, fumaric acid, tartaric acid, citric acid, 1-glutamic hydrochloride, maleic acid, and mixtures thereof; alternatively l-glutamic acid, lactic acid, citric acid, at a molar ratio of the amine to the acid of from about 1:0.3 to about 1:2, alternatively from about 1:0.4 to about 1:1.

### Mono-Long Alkyl Quaternized Ammonium Salt

The mono-long alkyl quaternized ammonium salts useful herein are those having one long alkyl chain which has from 12 to 30 carbon atoms, alternatively from 16 to 24 carbon atoms, alternatively a C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

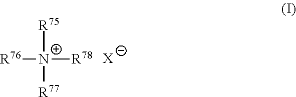

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, alternatively from 16 to 24 carbon atoms, alternatively from 18 to 22 carbon atoms, alternatively 22 carbon atoms; the remainder of R⁷⁵, R⁷⁶, R⁷⁷ and R⁷⁸ can be independently selected from CH₃, C₂H₅, C₂H₄OH, and mixtures thereof; and X can be selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Di-Long Alkyl Quaternized Ammonium Salts

When used, di-long alkyl quaternized ammonium salts can be combined with a mono-long alkyl quaternized ammonium salt and/or mono-long alkyl amine salt, at the weight ratio of from 1:1 to 1:5, alternatively from 1:1.2 to 1:5, alternatively from 1:1.5 to 1:4, in view of stability in rheology and conditioning benefits.

Di-long alkyl quaternized ammonium salts useful herein are those having two long alkyl chains of from 12 to 30 carbon atoms, alternatively from 16 to 24 carbon atoms, alternatively from 18 to 22 carbon atoms. Such di-long alkyl quaternized ammonium salts useful herein are those having the formula (I):

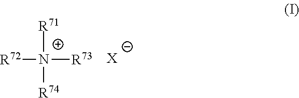

wherein two of R⁷¹, R⁷², R⁷³ and R⁷⁴ are selected from an aliphatic group of from 12 to 30 carbon atoms, alternatively from 16 to 24 carbon atoms, alternatively from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, alternatively from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C₁-C₄ alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Two of R⁷¹, R⁷², R⁷³ and R⁷⁴ can be selected from an alkyl group of from 12 to 30 carbon atoms, alternatively from 16 to 24 carbon atoms, alternatively from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Suitable di-long alkyl cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### High Melting Point Fatty Compound

The article further comprises a high melting point fatty compound at a level of from about 10 wt % to about 85 wt %, alternatively from 20 wt % to 70 wt %, alternatively from about 50 wt % to about 70 wt %, alternatively from about 10 wt % to about 20 wt % by weight of the article. The fatty compound can be selected from the group consisting of, but not limited to, fatty amphiphiles, fatty alcohol, fatty acid, fatty amide, fatty ester and combinations thereof.

The high melting point fatty compound useful herein have a melting point of 25° C. or higher, alternatively 40° C. or higher, alternatively 45° C. or higher, alternatively 50° C. or higher, in view of stability of the emulsion especially the gel matrix. Such melting point is up to about 90° C., alternatively up to about 80° C., alternatively up to about 70° C., alternatively up to about 65° C., in view of easier manufacturing and easier emulsification. The high melting point fatty compound can be used as a single compound or as a blend or mixture of at least two high melting point fatty compounds. When used as such blend or mixture, the above melting point means the melting point of the blend or mixture.

The high melting point fatty compound useful herein is selected from the group consisting of fatty alcohols, fatty acids, fatty alcohol derivatives, fatty acid derivatives, fatty amides, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than the above. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols can be used in the composition described herein. The fatty alcohols useful herein are those having from about 14 to about 30 carbon atoms, alternatively from about 16 to about 22 carbon atoms. These fatty alcohols are saturated and can be straight or branched chain alcohols.

Suitable fatty alcohols include, but are not limited to, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl or behenyl group.

Generally, in the mixture, the weight ratio of cetyl alcohol to stearyl alcohol is from about 1:9 to 9:1, alternatively from about 1:4 to about 4:1, alternatively from about 1:2.3 to about 1.5:1.

When using higher level of total cationic surfactant and high melting point fatty compounds, the mixture has the weight ratio of cetyl alcohol to stearyl alcohol of from about 1:1 to about 4:1, alternatively from about 1:1 to about 2:1, alternatively from about 1.2:1 to about 2:1, in view of maintaining acceptable consumer usage. It may also provide more conditioning on damaged part of the hair.

### Dispersing Agent

The article may further comprise a dispersing agent for increasing the wetting, hydration, and/or dispersion of the conditioner materials. The dispersing agent can be included at a level of from about 1 wt % to about 30 wt %, alternatively from about 5 wt % to about 15 wt %, and alternatively from about 5 wt % to about 10 wt % by weight of the second layer. A surfactant from the nonionic class of alkyl glucamides can improve the wetting and hydration when added to the solid conditioner formula. The alkyl glucamide surfactant contains a hydrophobic tail of about 8-18 carbons and a nonionic head group of glucamide. For glucamide, the presence of the amide and hydroxyl groups may provide sufficient polarity that balances the hydrophobic carbon tail in such a way to permit the surfactant's solubility in the conditioner oils and also imparts a rapid dispersion of the conditioner ingredients upon exposure to water. Other similar dispersing agents include, but are not limited to, reverse alkyl glucamides, gluconamides, cocoamiodpropyl betaines, alkyl glucoside, triethanol amine, cocamide MEAs and mixtures thereof.

### Plasticizer

The article may optionally comprise from about 0.1 wt % to about 25 wt % plasticizer, in one embodiment from about 3 wt % to about 20 wt % plasticizer, in one embodiment from about 5 wt % to about 15 wt % plasticizer. by weight of the layer.

When present in the articles, non-limiting examples of suitable plasticizing agents include polyols, copolyols, polycarboxylic acids, polyesters and dimethicone copolyols.

Examples of useful polyols include, but are not limited to, glycerin, diglycerin, propylene glycol, ethylene glycol, butylene glycol, pentylene glycol, cyclohexane dimethanol, hexane diol, polyethylene glycol (200-600), sugar alcohols such as sorbitol, manitol, lactitol, isosorbide, glucamine, N-methylglucamine and other mono- and polyhydric low molecular weight alcohols (e.g., C₂-C₈ alcohols); mono di- and oligo-saccharides such as fructose, glucose, sucrose, maltose, lactose, and high fructose corn syrup solids and ascorbic acid.

Examples of polycarboxylic acids include, but are not limited to citric acid, maleic acid, succinic acid, polyacrylic acid, and polymaleic acid.

Examples of suitable polyesters include, but are not limited to, glycerol triacetate, acetylated-monoglyceride, diethyl phthalate, triethyl citrate, tributyl citrate, acetyl triethyl citrate, acetyl tributyl citrate.

Examples of suitable dimethicone copolyols include, but are not limited to, PEG-12 dimethicone, PEG/PPG-18/18 dimethicone, and PPG-12 dimethicone.

Other suitable plasticizers include, but are not limited to, alkyl and allyl phthalates; napthalates; lactates (e.g., sodium, ammonium and potassium salts); sorbeth-30; urea; lactic acid; sodium pyrrolidone carboxylic acid (PCA); sodium hyraluronate or hyaluronic acid; soluble collagen; modified protein; monosodium L-glutamate; alpha & beta hydroxyl acids such as glycolic acid, lactic acid, citric acid, maleic acid and salicylic acid; glyceryl polymethacrylate; polymeric plasticizers such as polyquaterniums; proteins and amino acids such as glutamic acid, aspartic acid, and lysine; hydrogen starch hydrolysates; other low molecular weight esters (e.g., esters of C₂-C₁₀ alcohols and acids); and any other water soluble plasticizer known to one skilled in the art of the foods and plastics industries; and mixtures thereof.

EP 0283165 B1 discloses suitable plasticizers, including glycerol derivatives such as propoxylated glycerol.

### Optional Ingredients

The first and/or the second layer may comprise other optional ingredients that are known for use or otherwise useful in compositions, provided that such optional materials are compatible with the selected essential materials described herein, or do not otherwise unduly impair product performance.

Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1992.

Further non-limiting examples of such optional ingredients include preservatives, perfumes or fragrances, coloring agents or dyes, conditioning agents, hair bleaching agents, thickeners, moisturizers, emollients, pharmaceutical actives, vitamins or nutrients, sunscreens, deodorants, sensates, plant extracts, nutrients, astringents, cosmetic particles such as those containing perfumes and/or conditioning oils, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne agents, anti-microbial agents, sunscreens, tanning agents, exfoliation particles, hair growth or restorer agents, insect repellents, shaving lotion agents, co-solvents or other additional solvents, and similar other materials. Further non-limiting examples of optional ingredients include encapsulated perfumes, such as by □-cyclodetrins, polymer microcapsules, starch encapsulated accords and combinations thereof.

Suitable conditioning agents include high melting point fatty compounds, silicone conditioning agents and cationic conditioning polymers. Suitable materials are discussed in US 2008/0019935, US 2008/0242584 and US 2006/0217288.

### Physical Properties of the Dissolvable Article

For fibrous articles, the article comprises a significant number of dissolvable fibers with an average diameter less than about 150 micron, alternatively less than about 100 micron, alternatively less than about 10 micron, and alternatively less than about 1 micron with a relative standard deviation of less than 100%, alternatively less than 80%, alternatively less than 60%, alternatively less than 50%, such as in the range of 10% to 50%, for example. As set forth herein, the significant number means at least 10% of all the dissolvable fibers, alternatively at least 25% of all the dissolvable fibers, alternatively at least 50% of all the dissolvable fibers, alternatively at least 75% of all the dissolvable fibers. The significant number may be at least 99% of all the dissolvable fibers. Alternatively, from about 50% to about 100% of all the dissolvable fibers may have an average diameter less than about 15 micron. The dissolvable fibers produced by the method of the present disclosure have a significant number of dissolvable fibers with an average diameter less than about 1 micron, or sub-micron fibers. In an embodiment, Dissolvable article may have from about 25% to about 100% of all the dissolvable fibers with an average diameter less than about 1 micron, alternatively from about 35% to about 100% of all the dissolvable fibers with an average diameter less than about 1 micron, alternatively from about 50% to about 100% of all the dissolvable fibers with an average diameter less than about 1 micron, and alternatively from about 75% to about 100% of all the dissolvable fibers with an average diameter less than about 1 micron.

The percent porosity of the dissolvable article is at least about 25%, alternatively at embodiment at least about 50%, alternatively at least about 60%, alternatively at least about 70% and alternatively at least about 80%. The porosity of the dissolvable article is not more than about 99%, alternatively not more than about 98%, alternatively not more than about 95%, and alternatively not more than about 90%. Porosity of an article is determined according to the procedure set forth in the definition of “porosity” above.

A range of effective sizes of pores can be accommodated. The pore size distribution through the article cross-section may be symmetric or asymmetric.

The article can be flexible and have a distance to maximum force value of from about 6 mm to about 30 mm. The distance to maximum force value from about 7 mm to about 25 mm, alternatively from about 8 mm to about 20 mm, and alternatively from about 9 mm to about 15 mm.

The article can be characterized in one aspect by its Specific Surface Area. The article can have a Specific Surface Area of from about 0.03 m²/g to about 0.25 m²/g, alternatively from about 0.035 m²/g to about 0.22 m²/g, alternatively from about 0.04 m²/g to about 0.19 m²/g, and alternatively from about 0.045 m²/g to about 0.16 m²/g.

The article can be a flat, flexible article in the form of a pad, a strip, or tape and having a thickness of from about 0.5 mm to about 10 mm, alternatively from about 1 mm to about 9 mm, alternatively from about 2 mm to about 8 mm, and alternatively from about 3 mm to about 7 mm as measured by the below methodology. The article can be a sheet having a thickness from about 5 mm to about 6.5 mm. Alternatively, two or more sheets are combined to form an article with a thickness of about 5 mm to about 10 mm.

The article can have a basis weight of from about 75 grams/m² to about 2,000 grams/m², alternatively from about 400 g/m² to about 2,000 g/m², alternatively from about 600 g/m² to about 2,000 g/m², and alternatively from about 700 g/m² to about 2,000 g/m².

The article can have a dry density of from about 0.08 g/cm³ to about 0.50 g/cm³, alternatively from about 0.10 g/cm³ to about 0.38 g/cm³, alternatively from about 0.12 g/cm³ to about 0.35 g/cm³, and alternatively from about 0.15 g/cm³ to about 0.30 g/cm³.

### Method of Use

The dissolvable articles described herein may be used for cleaning and/or treating hair, hair follicles, skin, teeth, and the oral cavity. The method for treating these consumer substrates may comprise the steps of: a) applying an effective amount of the article to the hand, b) wetting the article with water to dissolve the solid, c) applying the dissolved material to the target consumer substrate such as to clean or treat it, and d) rinsing the diluted treatment composition from the consumer substrate. These steps can be repeated as many times as desired to achieve the desired cleansing and or treatment benefit.

A method useful for providing a benefit to hair, hair follicles, skin, teeth, and/or the oral cavity, includes the step of applying a composition according to the first embodiment to these target consumer substrates in need of regulating.

Alternatively, a useful method for regulating the condition of hair, hair follicles, skin, teeth, the oral cavity, includes the step of applying one or more compositions described herein to these target consumer substrates in need of regulation.

The amount of the composition applied, the frequency of application and the period of use will vary widely depending upon the purpose of application, the level of components of a given composition and the level of regulation desired. For example, when the composition is applied for whole body or hair treatment, effective amounts generally range from about 0.5 grams to about 10 grams, alternatively from about 1.0 grams to about 5 grams, and alternatively from about 1.5 grams to about 3 grams.

### Product Types and Articles of Commerce

Non-limiting examples of products that utilize the dissolvable articles include hand cleansing articles, teeth cleaning or treating articles, oral cavity articles, hair shampoo or other hair treatment articles, such as hair conditioner article, body cleansing articles, shaving preparation articles, personal care articles containing pharmaceutical or other skin care active, moisturizing articles, sunscreen articles, chronic skin benefit agent articles (e.g., vitamin-containing articles, alpha-hydroxy acid-containing articles, etc.), deodorizing articles, fragrance-containing articles, and so forth.

Preferably, the dissolvable article of the present disclosure is a personal care product, more preferably hair care product, still more preferably rinse-off hair care product, even more preferably rinse-off hair care product containing non-sulfate surfactant.

Described herein is an article of commerce comprising one or more dissolvable articles described herein, and a communication directing a consumer to dissolve the article and apply the dissolved mixture to hair, hair follicles, skin, teeth, the oral cavity, to achieve a benefit to the target consumer substrate, a rapidly lathering foam, a rapidly rinsing foam, a clean rinsing foam, and combinations thereof. The communication may be printed material attached directly or indirectly to packaging that contains the dissolvable article or on the dissolvable article itself. Alternatively, the communication may be an electronic or a broadcast message that is associated with the article of manufacture. Alternatively, the communication may describe at least one possible use, capability, distinguishing feature and/or property of the article of manufacture.

### Method of Forming Apertures

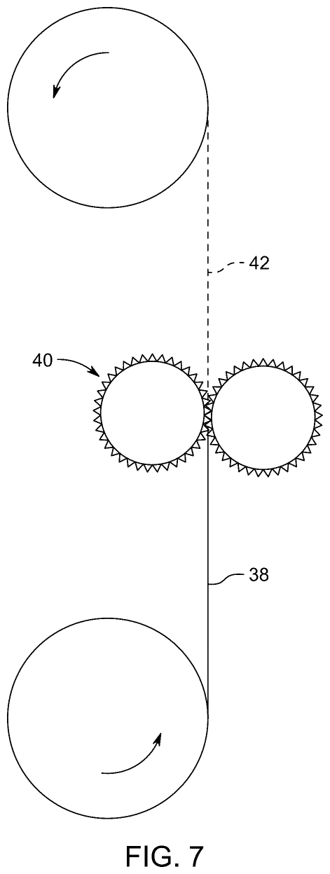

Once the precursor fibrous article (i.e., articles without apertures) has been formed, the precursor fibrous article may be subjected to an aperturing process; namely, a process that imparts one or more apertures to the fibrous article to produce an apertured fibrous article. Non-limiting examples of such aperturing processes include embossing, rodding, rotary knife aperturing, pinning, die cutting, die punching, needle punching, knurling, pneumatic forming, hydraulic forming, laser cutting, and tufting. FIG. 7 illustrates a non-limiting example of a suitable aperturing process. As shown in FIG. 7, a precursor fibrous article 38 is subjected to an aperturing operation (aperturing process) 40, non-limiting examples of such are described above, which results in one or more apertures being imparted to the precursor fibrous article 38 to form an apertured fibrous article 42.

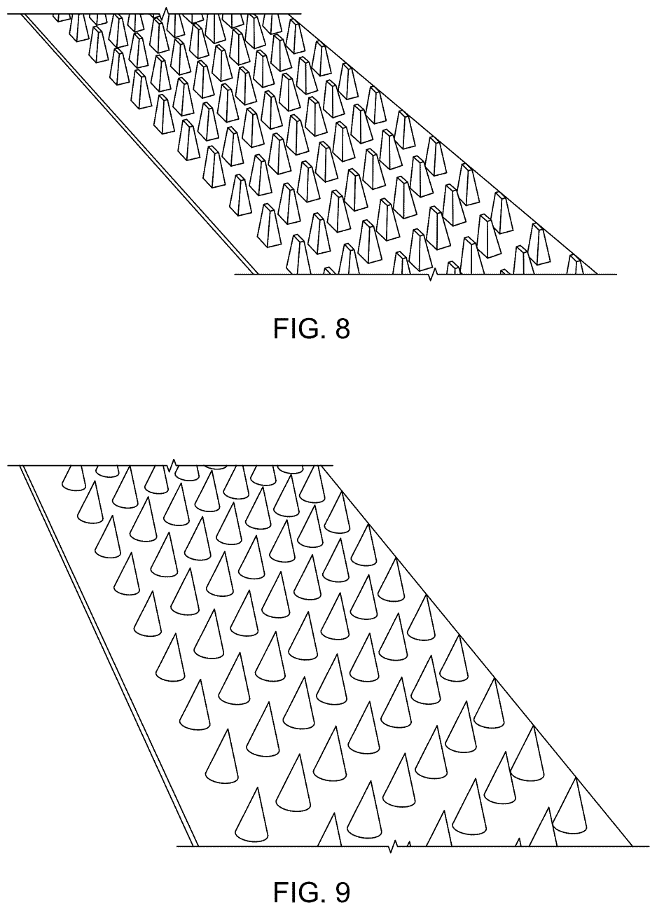

In one example, a precursor fibrous article is subjected to a rotary knife aperturing operation as generally described in U.S. Pat. No. 8,679,391. In this example of a suitable rotary knife aperturing operation for a web with stickiness as described herein, a precursor fibrous article is passed through a nip that comprises a 400 pitch toothed roll intermeshed with a second 400 pitch toothed roll. The teeth on the toothed roll have a pyramidal shape tip with four sides that taper from the base section to the tip of the tooth as shown in FIG. 8. The teeth are oriented so the long direction runs in the MD. The teeth are arranged in a staggered pattern, with a CD pitch P of 0.400 inch (10 mm) and a uniform tip to tip spacing in the MD is 0.400 inch (10 mm), the side wall angle on the long side of the tooth is 8.44 degrees and the side wall angle of the leading and trailing edges of the teeth in the pyramidal tip section is 8.44 degrees. The rotary knife aperturing rolls are aligned in the CD such that the clearances on either side of the teeth are about equal. The degree of interference between the virtual cylinders described by the tips of the teeth is described as the Depth of Engagement. As the fibrous article passes through the nip formed between the opposing rollers, the teeth from each roller engage with and penetrate the fibrous article to a depth determined largely by the depth of engagement between the rollers and the nominal thickness of the fibrous article.

With a fibrous article with the stickiness as described herein, this tooling may utilize a non-stick material, for example, silicone polymer, polyurethane, and blend of them. The rotary knife aperturing rolls may be fully comprised of this material or may utilize a metal base article with a coating of the non-stick material. The non-stick material may also be adhered or attached to a base metal cylinder. The tooth geometry may be formed by means such as milling, molding, or other known methods. The rolls and teeth may also be compliant to prevent compression of the fibrous article. In one example, the nonstick material has a shore A hardness of 90A.

In another example, the precursor fibrous article is subjected to a pinning operation as described below. In one example, the precursor fibrous article is passed through a nip that is formed between two opposing pin rollers of arranged in an intermeshing configuration so that pins from one roller pass through the space between pins on the opposing roller in the nip. A typical configuration may employ two rollers with the same pin design and arrangement. However, the opposing roller may be of a different pin design and arrangement, may instead not have pins, but other fibrous article support members, or may be a solid surface comprised of a compliant material allowing for interference between the pins of the pinned roller and the compliant surface. The opposing roller may contain indentations, holes, or grooves designed to receive the teeth from the knife side in an interpenetrating method. The degree of interference between the virtual cylinders described by the tips of the pins is described as the Depth of Engagement. As the fibrous article passes through the nip formed between the opposing rollers, the pins from each pinned roller engage with and penetrate the fibrous article to a depth determined largely by the depth of engagement between the rollers and the nominal thickness of the fibrous article. The pins used in the apparatus may be tapered pins having a circular cross section with a conical tip coming to a point as shown in FIG. 9. The maximum diameter of the pins, from the surface of the roll up to the base of the conical section is 0.118 inch. The conical section has a wall angle of 16.7 degrees. The total pin length extending above the surface of the roller is 0.197 inches (5 mm). The pins are arranged in staggered machine direction rows, each row of pins having an MD pitch (center to center) of 0.400 inches (10 mm) along the virtual circle described by the tips of the pins. Adjacent rows are spaced 0.400 inches (10 mm) in the cross direction and offset circumferentially by half the MD pitch. Opposing rollers are aligned such that the corresponding MD rows of each roller are in the same plane and such that the pins intermesh in a gear-like fashion with opposing pins passing near the center of the space between pins in the opposing roller MD row of pins.

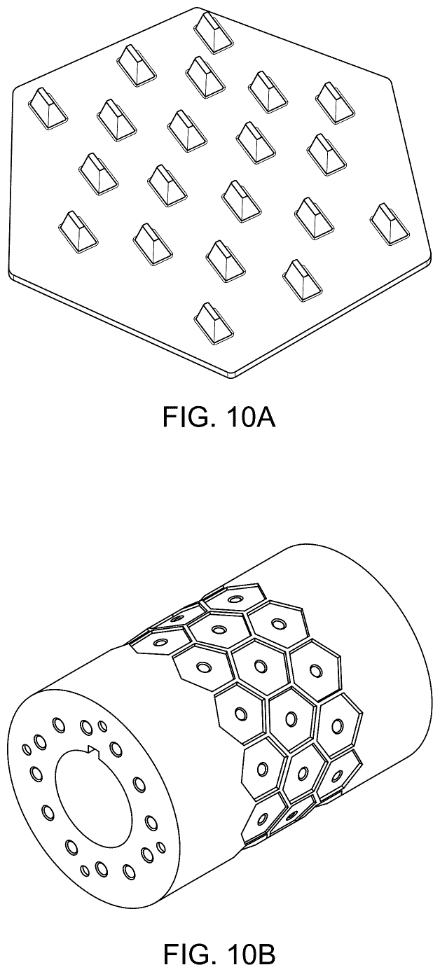

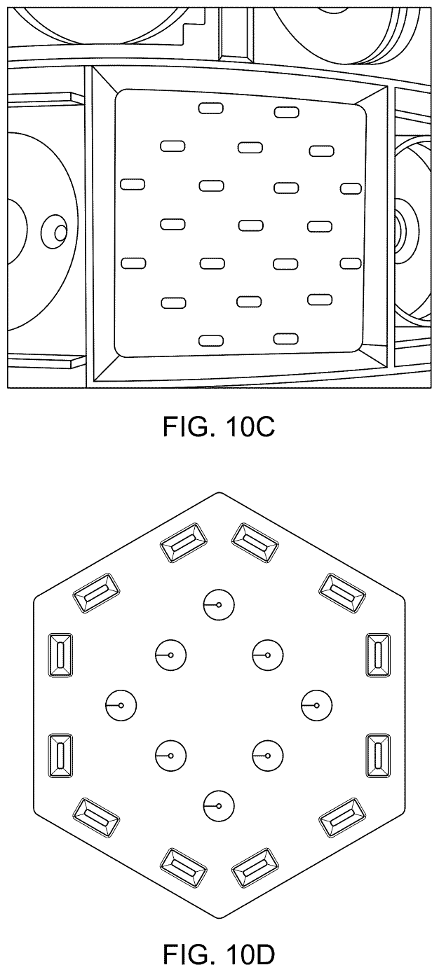

In another example, the precursor fibrous article is passed through a rotary aperturing and separation device. In this example, the fibrous article is passed through a device that provides apertures and discretizes a continuous web into articles concurrently. In one example, the precursor fibrous article is passed through a rotary die cutting nip that is formed between a cylindrical roller with cutting knives in a pattern outlining the shape of the article, and an anvil roller with smooth surfaces that mate against the knife pattern. The apertures are formed by pyramidal teeth which are discretely placed on the interior of the knife pattern and anvil pattern as shown in FIG. 10A. The teeth are arranged in a staggered pattern, with a CD pitch P of 0.400 inch (10 mm) and a uniform tip to tip spacing in the MD is 0.400 inch (10 mm), the side wall angle on the long side of the tooth is 16 degrees and the side wall angle of the leading and trailing edges of the teeth in the pyramidal tip section is 14.8 degrees by half the MD pitch. Teeth may also protrude from the interior of the anvil pattern, intermeshed with the tooth pattern on the knife roll. These teeth can be of the same size and shape and the anvil roll or may differ in height, angle or geometry such that the remain intermeshed. The anvil side may also be relatively flat or may contain indentations design to receive the teeth from the knife side in an interpenetrating method. As the fibrous article passes through the nip formed between the opposing rollers, the teeth engage with and penetrate the fibrous article to form the apertures while the die pattern on the knife roll compresses against the anvil roll to separate and discretize the article from the web.

An advantage of this example is to enable continuous centering or registration of the apertures relative to the edges of the article. The aperture pattern may be non-uniform to provide additional aesthetic of functional benefits to the article. The teeth or other shaped protrusions may vary in size based on the position in the article and relative to the edges of the article as shown in FIG. 10D. In one example, registering protrusions away from the cut edge may add in product dissolution and release from the equipment. The aperturing protrusions may also be arranged to form an aesthetic pattern of logo that is consistently placed and within the bounds of the article. A further advantage of this example is stabilization of the structure during aperturing and preservation of the desired aperture geometry throughout the article.

The knife and anvil rollers in this example may comprise pockets to receive inserts containing interchangeable tooth patterns which may be discretely attached within the perimeter of the knife and anvil patterns as shown in FIGS. 10B and 10C. The inserts may be mounted to the cylindrical rollers by adhesive, mechanical fastener, or snap-in mechanism such as an O-ring feature. With a fibrous article with the stickiness as described herein, the pattern of protrusions may be formed on an insert comprising a non-stick material, for example, silicone polymer, polyurethane, and a blend of them. The inserts may also be compliant to prevent compression of the fibrous article during the separation transformations, and allow the product to be more easily removed from the surface of the separation rollers. In one example, the nonstick material has a shore A hardness of 65A.

### Test Methods

### Aperture Characterization Test Method

Measurement of the Aperture Elliptical Length (AEL), the Aperture Elliptical Area (AEA), the Aperture Elliptical Percentage (AE %), the Fiber Elliptical Length (FEL) and Fiber Elliptical Area (FEA) is achieved using an optical magnification device. The optical magnification device is capable of between 0.6× to 20× magnification and is combined with a measuring scale, wherein the scale divisions include intervals of 0.1 mm. One such suitable optical magnification device is the Zeiss Stereo Microscope Stemi SV 11 Apo outfitted with a measuring scale reticle. The measuring scale reticle in the loupe is located at the focal plane of the lens. The loupe is placed in direct contact with the sample, thus allowing for precise measurement of aperture dimensions without parallax error or distortion. The transparent body of the loupe allows incident light to illuminate the sample. The selection of magnification is determined by the size of the aperture openings to be measured, since smaller aperture openings may require higher magnification than larger aperture openings.

The AEL and the FEL is a length value expressed in mm. The AEA and the FEA is an area value expressed in square mm. The AE % is a cumulative area value expressed as a percentage of the area of the planar surface which was inspected and in which the measured apertures were located.

For the purposes of this method, a single ply or single layer of a representative sample from the fibrous structure or dissolving article to be tested is laid out flat against a contrasting background and is inspected with sufficient oblique incident light and sufficient magnification to enable clear observation and measurement through the magnifying device, of the openings of individual apertures on the single ply or layer of web within fibrous structure or dissolving article. The openings of each aperture and the openings of the fibrous structure or dissolving article are measured at the upper most surface plane of the fibrous structure or dissolving article. This upper most surface plane is typically the location of the shoulder of the aperture opening, where the surface begins to dip downwards before forming the walls of the aperture. In some embodiments, the surrounding surface and shoulder of an aperture opening may occur at a plane which is elevated above (outward from) the generally planar surface of the fibrous structure or dissolving article, forming a volcano-like structure rising above the planar surface. In such cases, the aperture opening is measured at the outermost plane described by the perimeter of the aperture. In some cases, loose fibers from the article can impede or block the aperture after formation; these loose fibers are excluded from the measurement of the aperture opening. Furthermore, in some embodiments, the representative sample for conducting the method may be the actual dissolving article in its entirety. In such case, the above steps are followed except the measurements are performed on the intact, apertured article instead of a single ply or single layer of the fibrous structure or dissolving article.

Measurements are taken of the length of each individual aperture opening along two axes of that aperture or fiber opening. To conduct the two length measurements, a first length measurement is made along the major axis, which comprises the longest length of the aperture opening. A second length measurement is then made along the axis which is perpendicular to the previously measured major axis. Fiber Elliptical Length was measured only on the fibrous openings that are visible at 0.6× magnification, representing the largest void openings in the fibrous web structure.

For each aperture or fibrous opening,
the AEL and FEL value in that planar surface is used to calculate the AEA and FEA for that aperture in that planar surface, via the following equation for the area of an ellipse having the AEL or FEL value as its length:

$$AEA or FEA = π * {r}_{1} * {r}_{2}$$

$$where :$$

$$π = 3.1416$$

$$* Denotes the multiplication operator , and$$

$${r}_{1} = half the AEL or FEL value on the major axis$$

$${r}_{2} = half the AEL or FEL value on the mirror axis$$

The measurements of the length on two axis and area calculation are not limited to the shape of an aperture being an ellipse or apertures with rounded corners, but can be applied to any shape that has more than one length on an axis that can be measured. This can be applied to geometric shapes such as (but not limited to) circles, squares, rectangles, trapezoids, or triangles, where more than one axis is measured. The equation used to calculate the area for the aperture in that planar surface would be representative of the geometric shape, and is not limited to the area of an ellipse.

For fibrous structures or dissolving articles comprising a repeat pattern of apertures, the entire repeat pattern is inspected and all apertures with the inspected entire repeat pattern are measured as specified above. Sufficient replicates of the entire repeat pattern are inspected until all apertures within at least 10% of the total area of the entire fibrous structure or dissolving article have been measured. For fibrous structures or dissolving articles which lack a repeat pattern of apertures, at least 10% of the total area of the entire fibrous structure is inspected and all apertures within the inspected area are measured. The area(s) for inspection are selected such that the set of apertures measured is representative of the variety of apertures present and representative of their relative frequency in the structure (i.e., different aperture varieties are number-weighted, not area-weighted). Additionally, the rectangular area of the planar surface (A) which was inspected and in which the measured apertures were located is determined, and may be calculated from measurements obtained using a ruler or the measuring scale reticle in the magnification loupe A is calculated by measuring the length (L) and height (H) of the rectangular, using the equation

$$A = L * H$$

$$where :$$

$$* Denotes the multiplication operator , and$$

$$L = Length of planar surface measured$$

$$H = Height of planar surface measured$$

To determine the AE % value, all the AEA values measured for each planar surface are summed together to determine the cumulative area of the measured aperture openings on that planar surface. This cumulative area value is divided by the area of that planar surface which was inspected and in which the measured apertures were located. The result of that division is multiplied by 100 to yield the AE % value for that planar surface.

$$AE \% = \frac{{\sum }_{i = 1}^{n} {AEA}_{n}}{A}$$

$$where :$$

$${\sum }_{i = 1}^{n} = Summation of objects on surface from 1 ( initial ) to n ( final )$$

$${AEA}_{n} = Aperture Elliptical Area , representing numbers of objects from 1 ( initial ) to n ( final )$$

$$A = Area of planar surface$$

### “Stickiness Index” Test Method

The Stickiness Index is determined using the Stickiness Index Test Method. In this method, a specimen taken from an article(s) is held at an elevated temperature for an extended period of time in close proximity to an absorbent medium. The propensity of material from the article to melt, flow, and transfer to the absorbent medium is viewed as reflecting the dissolving article's stickiness and adherence to surface characteristics. The tendency for article to be subject to adhering or sticking to process surfaces during its making or manufacture may be reflected in the output of this method and its output parameter referred to as the Stickiness Index.

The ambient conditions of the laboratory are 23±2° C. and 40±10% relative humidity. Three sheets of 150-mm diameter Grade 4 filter paper (such as Whatman 1004-150, GE Healthcare Bio-Sciences, or equivalent) are stacked and the mass is recorded to within +0.01 g. This is the initial filter paper mass. The stack of filter paper is then placed onto a stainless steel support tray that extends beyond the 150-mm outer diameter of the filter paper. Solid spacing elements are placed beyond the edges of the filter papers to provide a gap above the filter paper. Spacing elements are preferably metal discs with a thickness of 8.5 mm+/−0.5 mm. A stainless steel grating is then placed on top of the stack of filter paper such that the filter paper is located between the two gratings (the grating assembly). The grating is composed of solid parallel rods 3.4 mm in diameter and spaced 12.5 mm on center in a perpendicular mesh planar configuration.

The mass of the specimen to be analyzed is 2.0±0.5 g, measured to within ±0.01 g. The specimen may be a full article with a surface area equal to or greater than 15 sq. cm. and having a minimum length of 20 mm in the shortest dimension. If this article weighs less than 1.5 g, additional articles are stacked until the total mass is 2.0±0.5 g, and this stack is the specimen used for the analysis. If the article has dimensions greater than 50 mm×50 mm, then a 50 mm×50 mm squares of material are cut from the center of the article and used as the specimen.

If a single article weighs more than 2.5 g, then a 50 mm×50 mm square of material is cut from the center of the article. If this 50 mm×50 mm square weighs 2.0±0.5 g, it is the specimen used for the analysis. If this 50 mm×50 mm square weighs more than 2.5 g, it is resampled in its center with a smaller square of mass 2.0±0.5 g, and this resulting smaller square is the specimen used for the analysis. (In this case, the final 50 mm×50 mm square used may be subsampled in its center in order to achieve the specified target mass of the specimen.) The specimen is then placed on the top grating such that it is centered over the stack of filter paper. This entire assembly is then placed (such that in an oven held at 90° C. for a duration of 3.0 hours. The racks are supported such that there is free space above the specimen and below the lower support tray where the 150 mm-diameter filter paper is positioned (that is, the lower support tray is not resting on the oven floor).

At the end of the 3-hour heating period, the grating assembly is removed from the oven, and the filter paper is removed from the between the assembly and allowed to re-equilibrate for 1 hour to ambient lab conditions. The mass of the filter paper along with any absorbed material from the article, defined as the final filter paper mass, is then determined to within +0.01 g. The Stickiness Index is calculated according to the equation below:

$$Stickiness Index = \frac{100 \% \times ( Final Filter Paper Mass - Initial Filter Paper Mass )}{Speciman Mass}$$

The Stickiness Index is reported as a percent rounded to the nearest integer percent value.

### Basis Weight Measurement

In general, basis weight of a material or article (including the dissolvable article) is measured by first cutting the sample to a known area, using a die cutter or equivalent, then measuring & recording the weight of the sample on a top-loading balance with a minimum resolution of 0.01 g, then finally by calculating the basis weight as follows:

$$Basis Weight ( g / {m}^{2} ) = weight of basis weight pad ( g )$$

$$Basis Weight ( \frac{g}{{m}^{2}} ) = \frac{Weight of pad ( g ) \times 10 , 000 \frac{{cm}^{2}}{{m}^{2}}}{area of pad ( {cm}^{2} )}$$

Suitable pad sample sizes for basis weight determination are >10 cm² and should be cut with a precision die cutter having the desired geometry. If the dissolvable article to be measured is smaller than 10 cm², a smaller sampling area can be sued for basis weight determination with the appropriate changes to calculation.

In the present examples, basis weight was calculated based on the full dissolvable article having a known area of 17.28 cm². Thus, the basis weight calculation becomes:

$$Basis Weight ( \frac{g}{{m}^{2}} ) = \frac{Weight of pad ( g ) \times 10 , 000 \frac{{cm}^{2}}{{m}^{2}}}{17.28 {cm}^{2}}$$

### Hand Dissolution Test Method

### Materials Needed:

Dissolvable articles to be tested: 3-5 dissolvable articles (finished product samples) are tested so that an average of the number of strokes for each if the individual dissolvable article samples is calculated and recorded as the Average Hand Dissolution value for the dissolvable article. For this method, the entire consumer saleable or consumer use dissolvable solid structure is tested. If the entire consumer saleable or consumer use dissolvable article has a footprint greater than 50 cm², then first cut the dissolvable article to have a footprint of 50 cm².
Nitrile Gloves
10 cc syringe
Plastic Weigh boat (~3 in ×3 in)
100 mL Glass beaker
Water (City of Cincinnati Water or equivalent having the following properties: Total Hardness=155 mg/L as CaCO2; Calcium content=33.2 mg/L; Magnesium content=17.5 mg/L; Phosphate content=0.0462 mg/L) Water used is 7 gpg hardness and 40° C.+/−5° C.

### Protocol:

1. Add 80 mL of water to glass beaker. Add 300-500 ml of water to glass beaker.
2. Heat water in beaker until water is at a temperature of 40° C.+/−5° C.
3. Transfer 10 mL of the water from the beaker into the weigh boat via the syringe.
4. Within 10 seconds of transferring the water to the weigh boat, place dissolvable article sample in palm of gloved hand (hand in cupped position in non-dominant hand to hold dissolvable article sample).
5. Using dominant hand, add water quickly from the weigh boat to the dissolvable article sample and allow to immediately wet for a period of 5-10 seconds.
6. Rub with opposite dominant hand (also gloved) in 2 rapid circular strokes.
7. Visually examine the dissolvable article sample in hand after the 2 strokes. If dissolvable article sample is completely dissolved, record number of strokes=2 Dissolution Strokes. If not completely dissolved, rub remaining dissolvable article sample for 2 more circular strokes (4 total) and observe degree of dissolution. If the dissolvable article sample contains no solid pieces after the 2 additional strokes, record number of strokes=4 Dissolution Strokes. If after the 4 strokes total, the dissolvable article sample still contains solid pieces of un-dissolved dissolvable article sample, continue rubbing remaining dissolvable article sample in additional 2 circular strokes and check if there are any remaining solid pieces of dissolvable article sample after each additional 2 strokes until dissolvable article sample is completely dissolved or until reaching a total of 30 strokes, whichever comes first. Record the total number of strokes. Record 30 Dissolution Strokes even if solid dissolvable article sample pieces remain after the maximum of 30 strokes.
8. Repeat this process for each of the additional 4 dissolvable article samples.
9. Calculate the arithmetic mean of the recorded values of Dissolution Strokes for the 5 individual dissolvable article samples and record as the Average Hand Dissolution Value for the dissolvable article. The Average Hand Dissolution Value is reported to the nearest single Dissolution Stroke unit.

### Fibrous Articles-Fiber Diameter

For fibrous articles, the diameter of dissolvable fibers in a sample of a web is determined by using a Scanning Electron Microscope (SEM) or an Optical Microscope and image analysis software. A magnification of 200 to 10,000 times is chosen such that the fibers are suitably enlarged for measurement. When using the SEM, the samples are sputtered with gold or a palladium compound to avoid electric charging and vibrations of the fibers in the electron beam. A manual procedure for determining the fiber diameters is used from the image (on monitor screen) taken with the SEM or the optical microscope. Using a mouse and a cursor tool, the edge of a randomly selected fiber is sought and then measured across its width (i.e., perpendicular to fiber direction at that point) to the other edge of the fiber. A scaled and calibrated image analysis tool provides the scaling to get actual reading in microns (μm). Several fibers are thus randomly selected across the sample of the web using the SEM or the optical microscope. At least two specimens from the web (or web inside a product) are cut and tested in this manner. Altogether at least 100 such measurements are made and then all data are recorded for statistical analysis. The recorded data are used to calculate average (mean) of the fiber diameters, standard deviation of the fiber diameters, and median of the fiber diameters. Another useful statistic is the calculation of the amount of the population of fibers that is below a certain upper limit. To determine this statistic, the software is programmed to count how many results of the fiber diameters are below an upper limit and that count (divided by total number of data and multiplied by 100%) is reported in percent as percent below the upper limit, such as percent below 1 micron diameter or %-submicron, for example. We denote the measured diameter (in microns) of an individual circular fiber as di.

In case the fibers have non-circular cross-sections, the measurement of the fiber diameter is determined as and set equal to the hydraulic diameter which is four times the cross-sectional area of the fiber divided by the perimeter of the cross of the fiber (outer perimeter in case of hollow fibers). The number-average diameter, alternatively average diameter is calculated as, dₙᵤₘ

$$\frac{{\sum }_{i = 1}^{n} {d}_{i}}{n}$$

### Combinations

1. A water-dissolvable fibrous article, wherein the dissolvable solid article is formed by a mixture comprising, by weight of the article:
from about 6% to about 30%, preferably from about 8% to about 23%, more preferably form about 10% to about 20% of a polymeric structurant;
from about 10 wt % to about 85 wt % of one or more high melting point fatty material having a carbon chain length C12-C22 or mixtures thereof, wherein the melting point is above 25° C.; and from about 1 wt % to about 60 wt % of a cationic surfactant;
wherein the water-dissolvable fibrous article has one or more apertures which exhibit at least one of the following properties, preferably both of the following properties:
a) an Aperture Elliptical Length of from about 0.1 mm to about 10 mm, preferably from about 0.2 mm to about 6 mm, as measured according to the Aperture Characterization Test Method described herein; and
b) an Aperture Elliptical Area of from about 0.5 mm² to about 15 mm², preferably from about 0.5 mm² to about 7 mm² as measured according to the Aperture Characterization Test Method described herein.

2. The article of the preceding feature, wherein the polymeric structurant is one selected from polyvinylpyrrolidone, its copolymers, and combinations thereof.

3. The article of any of the preceding feature, wherein the polymeric structurant is selected from the group consisting of: polyacrylic acid and its copolymers, polyacrylamide and its copolymers, polyvinylmethyl ether, polyethyleneimine, polymethacrylic acid, polyN-isopropyl acrylamide, polyN-N-dimethylacrylamide, polyvinyl oxazoline, polyaryl oxazoline, polyalkyl oxazoline, poly(2-ethyl-2-oxazoline), polyoxazoline, polyvinyloxazolidone, polyvinyl caprolactam, polystyrene sulfonate, polyvinyl formamide, polyvinyl amine, alkylated polyvinyl pyrrolidone, poly vinyl caprolactam, polyvinyl valerolactam, polyvinyl imidazole, polyacrylic acid, polyacrylamide, polymethacrylamide, polydimethacrylamide, polyalkylaminomethacrylate, and polyalkylaminomethacrylamide, and combinations thereof, preferably the polymeric structurant is selected from the group consisting of: polydimethylacrylamide, polyalkyl oxazoline, poly(2-ethyl-2 oxazoline), polyaryl oxazoline, and combinations thereof.

4. The article of any of the preceding features, wherein the apertures exhibit the following additional property:
c) an Aperture Elliptical Percentage of from about 0.1% to about 50%, preferably from about 0.5% to about 20%, as measured according to the Aperture Characterization Test Method described herein.

5. The article of any of the preceding features, wherein the article exhibits a Stickiness Index of from about 20% to about 100%, preferably from about 30% to about 100%, more preferably from about 40% to about 100%, still more preferably from about 50% to about 100%, even more preferably from about 60% to about 99% as measured according to the Stickiness Index Test Method described herein.

6. The article of any of the preceding features, where article is comprised of more than 1 ply with the apertures formed through article surfaces.

7. The article of any of the preceding features, where apertures are formed in a pattern registered to the shape of the article.

8. The article of any of the preceding features, where apertures are formed by a non-sticky material selected from the group consisting of: silicone polymer, polyurethane, and blend of them, and wherein the material has a Shore A hardness of 95 or below.

9. The article of any of the preceding features, having a basis weight of 800 gsm or more, preferably 1400 gsm or more.

10. The article of any of the preceding features, comprising from about 40 wt % to about 65 wt % of one or more high melting point fatty alcohol material having a carbon chain length C12-C22 or mixtures thereof, wherein the melting point is above 25° C.; and from about 15 wt % to about 30 wt % of a cationic surfactant.

11. The article of any of the preceding features, wherein the article dissolves in less than 15 strokes, preferably less than 10 strokes of the Hand Dissolution Method.

12. The article of any of the preceding features, wherein the article contains a particulate material, wherein the particulate material is integrated into the fibrous structure, and/or is between plies of the article.

### Examples

The articles illustrated in the following Examples illustrate specific embodiments of the articles according to the present disclosure, but are not intended to be limiting thereof. Other modifications can be undertaken by the skilled artisan without departing from the spirit and scope of this invention.

All exemplified amounts are listed as weight percents and exclude minor materials such as diluents, preservatives, color solutions, imagery ingredients, botanicals, and so forth, unless otherwise specified. All percentages are based on weight unless otherwise specified.

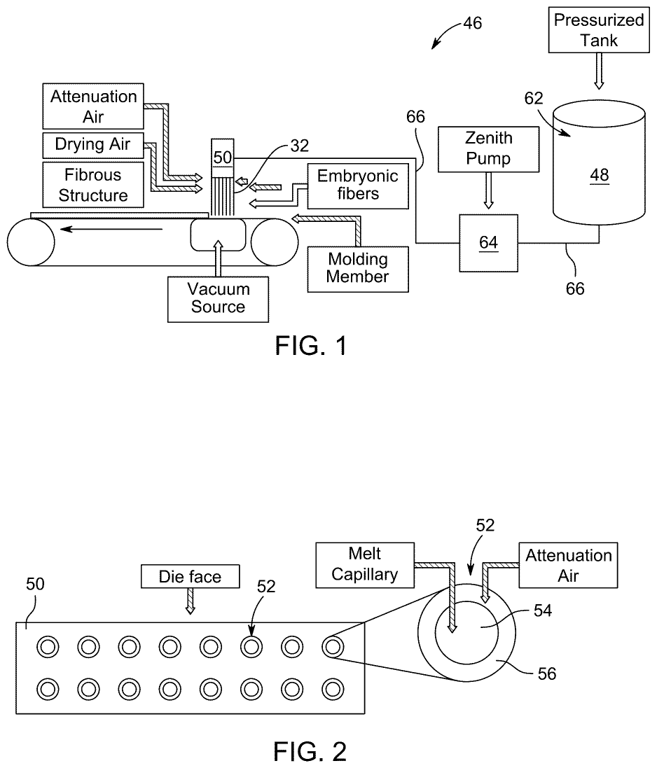

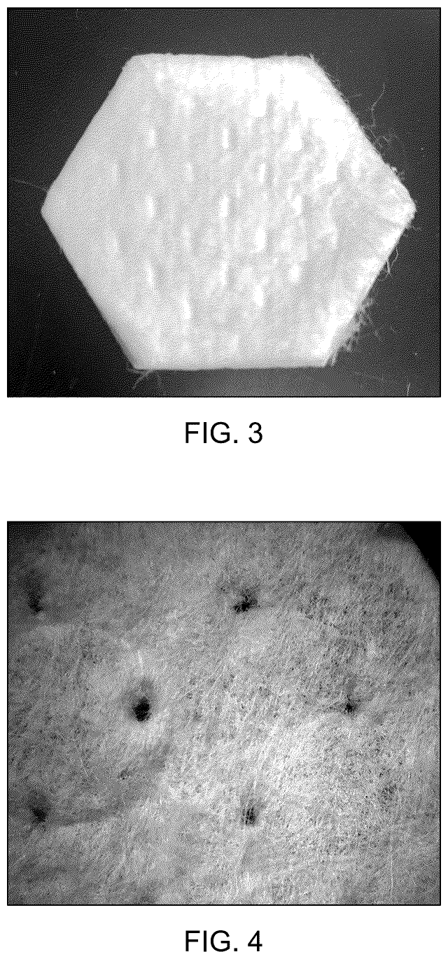

The precursor fibrous articles (i.e., articles without apertures) used in the examples are made as described hereinabove, for example as shown in FIGS. 1 and 2.

Apertured Article Example 1 (Ex. 1), shown in FIG. 4

| | % | |
|---|---|---|
| Ex. 1 formulation | | |
| Polyvinyl pyrrolidone | 10.7 | |
| (PVP K30 from Ashland) having MW of 66,800 | | |
| Polyvinyl pyrrolidone | 1.6 | |
| (PVP K60 from Jarchem) having MW of 396,000 | | |
| Polyvinyl pyrrolidone | 4.0 | |
| (PVP K120 from Ashland) having MW of 3,470,000 | | |
| Behentrimonium Methosulfate | 24.8 | |
| Stearyl Alcohol | 36.6 | |
| 1-Hexadecanol | 14.9 | |
| Lauroyl Methyl Glucamide | 7.4 | |
| Ex. 1 article properties | | |
| Aperture Elliptical Length | 0.51 | mm |
| Aperture Elliptical Area | 0.22 | mm² |
| Aperture Elliptical Percentage | 0.52% | |
| Stickiness index | 76.8% | |
| Basis weight | 1500 | gsm |

A precursor fibrous article comprising a plurality of filaments is prepared and layered in stacks of 3 plies (forming a precursor multi-ply fibrous article, in other words prior to aperturing), 175 mm wide, aligned with the CD of the precursor fibrous article, wherein each ply has a nominal basis weight of 500 gsm and a thickness of approximately 1.5 mm. The precursor multi-ply fibrous article is then apertured a pinning operation by passing through a nip that is formed between two opposing rollers arranged in an intermeshing configuration so that pins from one roller pass through the space between pins on the opposing roller in the nip as shown in FIG. 7. The rollers are covered with a non-stick material with a base thickness of 1 mm. The pins used in the apparatus are tapered pins having a circular cross section with a conical tip coming to a point as shown in FIG. 9. The maximum diameter of the pins, from the surface of the roll up to the base of the conical section is 0.118 inch. The conical section has a wall angle of 16.7 degrees. The total pin length extending above the surface of the roller is 0.197 inches (5 mm). The pins are arranged in staggered machine direction rows, each row of pins having an MD pitch (center to center) of 0.400 inches (10 mm) along the virtual circle described by the tips of the pins. Adjacent rows are spaced 0.400 inches (10 mm) in the cross direction and offset circumferentially by half the MD pitch. Opposing rollers are aligned such that the corresponding MD rows of each roller are in the same plane and such that the pins intermesh in a gear-like fashion with opposing pins passing near the center of the space between pins in the opposing roller MD row of pins. The depth of engagement between the toothed and ring rolls is set to about 0.020 inches. The precursor multi-ply fibrous article is passed through the nip with essentially zero wrap around the rolls both ingoing and outgoing.

The multi-ply fibrous article is passed through the nip at a speed of about 15 meters per minute. The resulting apertured multi-ply fibrous article is cut into polygonal shapes of an area of approximately 17 sq. cm.

Observation of the apertures in the resulting apertured multi-ply fibrous article using the Aperture Characterization Test Method described herein revealed well-formed, generally circular apertures, each aperture having a larger opening disposed towards one planar surface of the fibrous article and a smaller opening disposed towards the opposite fibrous article All apertures are disposed such that the larger opening was towards the same generally planar surface, referred as the “tooth-side” surface. The typical aperture exhibits an Aperture Elliptical Length at the “tooth-side” surface of 0.4-0.8 mm.

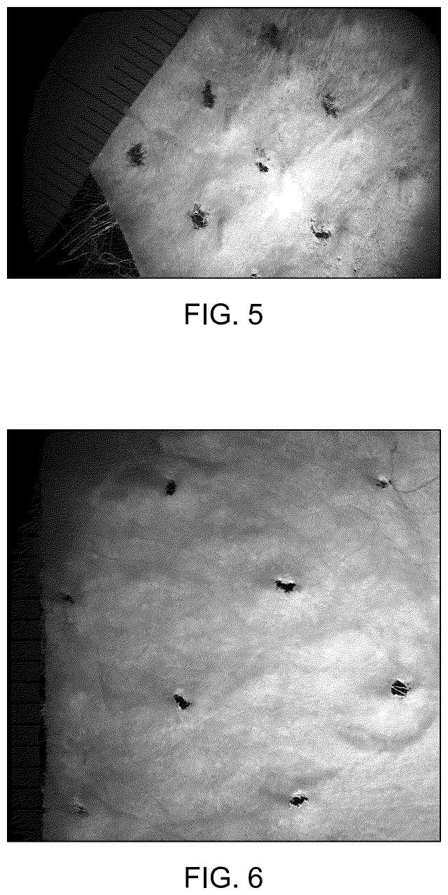

Apertured Article Example 2 (Ex. 2), shown in FIG. 5

| | % | |
|---|---|---|
| Ex. 2 formulation | | |
| Polyvinyl pyrrolidone | 10.7 | |
| (PVP K30 from Ashland) having MW of 66,800 | | |
| Polyvinyl pyrrolidone | 1.6 | |
| (PVP K60 from Jarchem) having MW of 396,000 | | |
| Polyvinyl pyrrolidone | 4.0 | |
| (PVP K120 from Ashland) having MW of 3,470,000 | | |
| Behentrimonium Methosulfate | 24.8 | |
| Stearyl Alcohol | 36.6 | |
| 1-Hexadecanol | 14.9 | |
| Lauroyl Methyl Glucamide | 7.4 | |
| Ex. 2 article properties | | |
| Aperture Elliptical Length | 1.37 | mm |
| Aperture Elliptical Area | 1.38 | mm² |
| Aperture Elliptical Percentage | 4.47% | |
| Stickiness index | 69.1% | |
| Basis weight | 1050 | gsm |

A precursor fibrous article comprising a plurality of filaments is prepared and layered in stacks of 3 plies (forming a precursor multi-ply fibrous article, in other words prior to aperturing), 175 mm wide, aligned with the CD of the precursor fibrous article, wherein each ply has a nominal basis weight of 350 gsm and a thickness of approximately 1.5 mm.

The precursor multi-ply fibrous article is then apertured by passing the multi-ply fibrous article through a nip of a rotary knife aperturing device that comprises a 400 pitch toothed roll intermeshed with a second 400 pitch toothed roll as shown in FIG. 7 and as described further below. The teeth on the toothed roll have a pyramidal shape tip with four sides that taper from the base section to the tip of the tooth. The teeth are oriented so the long direction runs in the MD and are arranged in a staggered pattern, with a CD pitch P of 0.400 inch (10 mm) and a uniform tip to tip spacing in the MD is 0.400 inch (10 mm), the side wall angle on the long side of the tooth is 8.44 degrees and the side wall angle of the leading and trailing edges of the teeth in the pyramidal tip section is 8.44 degrees. The rotary knife aperturing rolls are aligned in the CD such that the clearances on either side of the teeth are about equal. The degree of interference between the virtual cylinders described by the tips of the teeth is described 0.200 inch (5 mm). As the fibrous article passes through the nip formed between the opposing rollers, the teeth from each roller engage with and penetrate the fibrous article to a depth determined largely by the depth of engagement between the rollers and the nominal thickness of the fibrous article.

Observation of the apertures in the resulting apertured multi-ply fibrous article using the Aperture Characterization Test Method described herein revealed well-formed, generally elongate apertures, each aperture having a larger opening disposed towards one planar surface of the fibrous article and a smaller opening disposed towards the opposite fibrous article All apertures are disposed such that the larger opening was towards the same generally planar surface, referred as the “tooth-side” surface. The typical aperture exhibits an Aperture Elliptical Length at the “tooth-side” surface of 1.0-1.8 mm and an Aperture Elliptical Area of 1.0-1.6 mm².

Apertured Article Example 3 (Ex. 3), shown in FIG. 6

| | % | |
|---|---|---|
| Ex. 3 formulation | | |
| Polyvinyl pyrrolidone | 8.025 | |
| (PVP K30 from Ashland) having MW of 66,800 | | |
| Polyvinyl pyrrolidone | 1.2 | |
| (PVP K60 from Jarchem) having MW of 396,000 | | |
| Polyvinyl pyrrolidone | 3 | |
| (PVP K120 from Ashland) having MW of 3,470,000 | | |
| Behentrimonium Methosulfate | 16.35 | |
| Stearyl Alcohol | 28.8 | |
| 1-Hexadecanol | 11.775 | |
| Lauroyl Methyl Glucamide | 5.85 | |
| Encapsulated Fragrance Particles | 25 | |
| Ex. 3 article properties | | |
| Aperture Elliptical Length | 0.80 | mm |
| Aperture Elliptical Area | 0.48 | mm² |
| Aperture Elliptical Percentage | 0.97% | |
| Stickiness index | 70.9% | |
| Basis weight | 1200 | gsm |

A precursor fibrous article comprising a plurality of filaments and one or more particulate materials is prepared and layered in stacks of 3 plies (forming a precursor multi-ply fibrous article, in other words prior to aperturing), 175 mm wide, aligned with the CD of the precursor fibrous article, wherein each ply has a nominal basis weight of 400 gsm and a thickness of approximately 1.5 mm

The precursor multi-ply fibrous article is then apertured a pinning operation by passing through a nip that is formed between two opposing rollers arranged in an intermeshing configuration so that pins from one roller pass through the space between pins on the opposing roller in the nip as shown in FIG. 7. The rollers are covered with a non-stick material with a base thickness of 1 mm. The pins used in the apparatus are tapered pins having a circular cross section with a conical tip coming to a point as shown in FIG. 9. The maximum diameter of the pins, from the surface of the roll up to the base of the conical section is 0.118 inch. The conical section has a wall angle of 16.7 degrees. The total pin length extending above the surface of the roller is 0.197 inches (5 mm). The pins are arranged in staggered machine direction rows, each row of pins having an MD pitch (center to center) of 0.400 inches (10 mm) along the virtual circle described by the tips of the pins. Adjacent rows are spaced 0.400 inches (10 mm) in the cross direction and offset circumferentially by half the MD pitch. Opposing rollers are aligned such that the corresponding MD rows of each roller are in the same plane and such that the pins intermesh in a gear-like fashion with opposing pins passing near the center of the space between pins in the opposing roller MD row of pins. The depth of engagement between the toothed and ring rolls is set to about 0.020 inches. The precursor multi-ply fibrous article is passed through the nip with essentially zero wrap around the rolls both ingoing and outgoing.

The multi-ply fibrous article is passed through the nip at a speed of about 12 meters per minute. The resulting apertured multi-ply fibrous article is cut into polygonal shapes of an area of approximately 6 sq. cm.

Observation of the apertures in the resulting apertured multi-ply fibrous article using the Aperture Characterization Test Method described herein revealed well-formed, generally circular apertures, each aperture having a larger opening disposed towards one planar surface of the fibrous article and a smaller opening disposed towards the opposite fibrous article All apertures are disposed such that the larger opening was towards the same generally planar surface, referred as the “tooth-side” surface. The typical aperture exhibits an Aperture Elliptical Length at the “tooth-side” surface of 0.6-1.2 mm.

As shown in Ex. 1-3, the present disclosure provides water-dissolvable fibrous articles with apertures, even when the article may exhibit sticky behavior, for example, articles sticking to surfaces of process equipment during their making. With such apertures, the articles of the present disclosure can provide improved dissolution and/or improved water penetration into the article, especially for articles with increased size or weight, for example, improve dissolution of less than 10 hand strokes for the article with increased basis weight of 800 gsm or more, preferably 1400 gsm or more. Additionally, the water-dissolvable fibrous articles of the present inventing may exhibit reduced tendency to stick to surfaces of process equipment during the aperture formation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A water-dissolvable fibrous article, wherein the article is formed by a mixture comprising, by weight of the article:
from about 6% to about 30% of a polymeric structurant;
from about 10 wt % to about 85 wt % of one or more high melting point fatty material having a carbon chain length C12-C22 or mixtures thereof, wherein the melting point is above 25° C.; and
from about 1 wt % to about 60 wt % of a cationic surfactant;
wherein the water-dissolvable fibrous article has one or more apertures which exhibit at least one of the following properties:
a) an Aperture Elliptical Length of from about 0.1 mm to about 10 mm, as measured according to the Aperture Characterization Test Method described herein; and/or
b) an Aperture Elliptical Area of from about 0.5 mm² to about 15 mm², as measured according to the Aperture Characterization Test Method described herein.

2. The article of claim 1, wherein the one or more apertures exhibit both of the following properties:
a) an Aperture Elliptical Length of from about 0.1 mm to about 10 mm, as measured according to the Aperture Characterization Test Method described herein; and
b) an Aperture Elliptical Area of from about 0.5 mm² to about 15 mm², as measured according to the Aperture Characterization Test Method described herein.

3. The article of claim 1, wherein the article is formed by a mixture comprising, by weight of the article, from about 8% to about 23%, of the polymeric structurant.

4. The article of claim 1, wherein the polymeric structurant is chosen from polyvinylpyrrolidone, its copolymers, or mixtures thereof.

5. The article of claim 1, wherein the polymeric structurant is chosen from polyacrylic acid and its copolymers, polyacrylamide and its copolymers, polyvinylmethyl ether, polyethyleneimine, polymethacrylic acid, polyN-isopropyl acrylamide, polyN-N-dimethylacrylamide, polyvinyl oxazoline, polyaryl oxazoline, polyalkyl oxazoline, poly(2-ethyl-2-oxazoline), polyoxazoline, polyvinyloxazolidone, polyvinyl caprolactam, polystyrene sulfonate, polyvinyl formamide, polyvinyl amine, alkylated polyvinyl pyrrolidone, poly vinyl caprolactam, polyvinyl valerolactam, polyvinyl imidazole, polyacrylic acid, polyacrylamide, polymethacrylamide, polydimethacrylamide, polyalkylaminomethacrylate, and polyalkylaminomethacrylamide, or mixtures thereof.

6. The article of claim 5, wherein the polymeric structurant is chosen from polydimethylacrylamide, polyalkyl oxazoline, poly(2-ethyl-2 oxazoline), polyaryl oxazoline, or mixtures thereof.

7. The article of claim 1, wherein the Aperture Elliptical Length is from about 0.2 mm to about 6 mm, as measured according to the Aperture Characterization Test Method.

8. The article of claim 1, wherein the Aperture Elliptical Area is from about 0.5 mm² to about 7 mm², as measured according to the Aperture Characterization Test Method.

9. The article of claim 1, wherein the apertures have an Aperture Elliptical Percentage of from about 0.1% to about 50%, as measured according to the Aperture Characterization Test Method.

10. The article of claim 9, wherein the Aperture Elliptical Percentage is from about 0.5% to about 20% as measured according to the Aperture Characterization Test Method.

11. The article of claim 1, wherein the article exhibits a Stickiness Index of from about 20% to about 100%, as measured according to the Stickiness Index Test Method.

12. The article of claim 11, wherein the Stickiness Index is from about 50% to about 100%, as measured according to the Stickiness Index Test Method.

13. The article of claim 1, where article is comprised of more than 1 ply with the apertures formed through article surfaces.

14. The article of claim 1, where apertures are formed in a pattern registered to the shape of the article.

15. The article of claim 1, where apertures are formed by a non-sticky material chosen from silicone polymer, polyurethane, or mixtures thereof; and wherein the non-sticky material has a Shore A hardness of 95 or below.

16. The article of claim 1, wherein the article has a basis weight of 800 gsm or more.

17. The article of claim 16, wherein the basis weight is 1400 gsm or more.

18. The article of claim 1, comprising from about 40 wt % to about 65 wt % of one or more high melting point fatty alcohol material having a carbon chain length C12-C22 or mixtures thereof; wherein the melting point is above 25° C.; and from about 15 wt % to about 30 wt % of a cationic surfactant.

19. The article of claim 1, wherein the article dissolves in less than 15 strokes of the Hand Dissolution Method.

20. The article of claim 1, wherein the article contains a particulate material, wherein the particulate material is integrated into the fibrous structure, and/or is between plies of the article.
